(19) United States
(12) Patent Application Publication
(10) Pub. No.: US 2025/0152076 A1
(43) Pub. Date: May 15, 2025

(54) Identifying Gifted Students through Analysis of Brain Activity Energy Emission and Nerve Impulse Transmission Speed between Brain Cells

(71) Applicant: King Faisal University, AL-AHSA (SA)

(72) Inventors: MAMDOUH MOSAAD AHMED HELALI, AL-AHSA (SA); ROMMEL MAHMOUD AL ALI, AL-AHSA (SA); MOHAMMED AL QAHTANI, AL-AHSA (SA)

(21) Appl. No.: 18/389,509

(22) Filed: Nov. 14, 2023

(51) Int. Cl. A61B 5/374; A61B 5/384

(52) U.S. Cl. CPC A61B 5/374; A61B 5/384

(57) ABSTRACT

Identification and categorizing of people according to educational capability is performed as indicated by detected brain activity by use of a Brain-Computer Interface (BCI) and associated sensor electrodes. Locations on a subject's head for detection of brain electrical activity emanating from a brain region most associated with creativity are identified, and the sensor electrodes are positioned on the subject's head at positions to allow the detection of the brain electrical activity at those locations. An output of the BCI is provided to a monitor, and the subject's brain activity is monitored with the BCI connected to the sensor electrodes. Sensed quantity and speed of change measurements of brain electrical activity emanating from the brain region most associated with creativity using the BCI are monitored according to the sensed quantity and speed of change measurements.

### BACKGROUND

### Technical Field

The present disclosure relates generally to measurement of brain electrical activity emanating from the brain region most associated with creativity using a brain computer interface (BCI).

### Background Art

It is desired to uncover and nurture gifted students to enhance the effectiveness of the education system, reduce expenses associated with unsuitable study programs, and replace them with high-quality programs tailored to students' cognitive abilities.

U.S. Patent Application 2023/0025019 A1 to Youngblood, et al. describes features related to measuring the energy emitted by brain cells. This includes the use of a brain computer interface (BCI), which converts a representation of measured electrical activity into a color spectrum using Kirlian imaging/photography. That technique involves measuring the mental energy generated while individuals engage in problem-solving exercises. The approach is one of quantifying the amount of emitted energy from the brain.

### SUMMARY

Identification and categorizing of people according to educational capability is performed as indicated by detected brain activity. A Brain-Computer Interface (BCI) and associated sensor electrodes are provided. Locations on a subject's head for detection of brain electrical activity emanating from a brain region most associated with creativity are identified, and the sensor electrodes are positioned on the subject's head at positions to allow the detection of the brain electrical activity at those locations. An output of the BCI is provided to a monitor, and the subject's brain activity is monitored with the BCI connected to the sensor electrodes. Sensed quantity and speed of change measurements of brain electrical activity emanating from the brain region most associated with creativity using the BCI are monitored according to the sensed quantity and speed of change measurements.

In one configuration, the monitored BCI measurement provides a determination of a speed of transmission of neurological signals within the brain, by detection of the speed of transmission as sensed by different ones of the sensors. A correlation is made of the detection of the speed of transmission with neural activity associated with the subject's cognitive processing. The correlation provides a means to categorize or measure intelligence.

### BRIEF DESCRIPTION OF THE DRAWINGS

FIG. 1 is a schematic diagram showing the configuration of the disclosed technique.

### DETAILED DESCRIPTION

The present disclosure relates generally to measurement of brain electrical activity emanating from the brain region most associated with creativity using a brain computer interface (BCI) which converts the measured electrical activity representation into a color spectrum using Kirlian imaging/photography.

An electronic device utilizes the detection and analysis of brain activity energy, particularly during cognitive tasks aimed at solving innovative problems. The device measures and analyzes the emitted energy derived from students' brain activity, allowing for the classification of students into gifted and non-gifted categories based on the quantity of emitted energy. The energy is sensed using a brain computer interface (BCI) which converts the measured electrical activity representation into a color spectrum using Kirlian imaging/photography. The measurement protocol includes measuring students emitted mental energy generated during exercises in problem solving where presented problems are used as diagnostic tools in ascertaining an individual's intelligence. The emitted mental energy is a direct correlation of measured quantity and velocity of electrical energy emitted during cognitive processing and the results are used to categorize measured intelligence as “gifted”, “average”, and “below average”.

The concept behind the device bears resemblance to various technologies, such as the device employed to record the thoughts of physicist Stephen William Hawking. Additionally, the concept shares similarities with diagnostic recognition devices that utilize eye prints and lie detectors. The present technique draws parallels to the imaging of electro-biological energy emitted from the human body, known as the optical aura, which is captured through the use of a camera (Kirlian photography). This energy is represented by a spectrum of colors, by way of non-limiting example, ranging from red (indicating vitality) to blue (indicating illness within the body).

In use, the student or other subject is exposed to a sensitive part of the device designated to detect and analyze the energy emitted from mental activity while contemplating a simple creative problem the subject aims to solve. The presented problems vary in difficulty level, spanning from easy to challenging. After each instance, the emitted mental energy is measured and analyzed, allowing for the identification of the subject's cognitive capabilities, which serves as an indicator of the subject's creativity. Based on the analysis of the subject's mental energy, the subject can be categorized as normal, moderate, or high in terms of creative abilities.

The use of this technique provides a reduction of educational setbacks arising from the placement of gifted students in programs tailored for average students. By transferring them to programs that align with their skills and creative abilities, the educational loss is minimized. This approach ensures that gifted students receive appropriate educational opportunities, enabling them to flourish and reach their full potential.

The technique can be further enhanced by leveraging artificial intelligence technology and computer programs capable of predicting users' ideas and providing tailored recommendations based on their preferences even before they articulate them explicitly. This advancement enables a proactive and personalized user experience, catering to individual desires and needs.

A significant distinction over prior techniques is in the methodology involved. According to the present technology, a dual approach, combining quantity and speed of change measurements, enhances the accuracy and reliability of the results. The present disclosure focuses on the measurement of both the quantity and speed of changes of electrical energy emissions from the brain, whereas previous attempts relied on solely quantifying the amount of emitted energy. This dual approach, combining quantity and speed of change measurements, enhances the accuracy and reliability of the results. The correlation between the amount of electrical energy and the speed of changes of energy transmission between brain cells enhances the overall validity of the findings. Additionally, the BCI measurements, as monitored, can be used to determine a speed of transmission of neurological signals within the brain. By way of non-limiting example, the speed of transmission may be sensed by a time difference between sensing by different ones of the sensors. This detection may be correlated with neural activity associated with the subject's cognitive processing and the results used to categorize measured intelligence.

Thus, the current technique concentrates on quantifying the quantity and speed of changes of electrical energy emitted from the brain exclusively during cognitive problem-solving. This is used to classify individuals into categories such as, by way of non-limiting example, “gifted,” “average,” and “below average”.

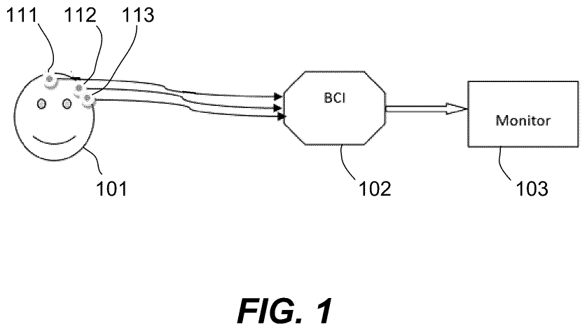

FIG. 1 is a schematic diagram showing the configuration of the disclosed technique. Depicted is the subject 101, a Brain-Computer Interface (BCI) 102 and a monitor 103. BCI 102 is connected to a plurality of sensors 111, 112, 113, from which BCI 102 receives EEG brain signals.

The measurement of brain electricity emanating from the region responsible for creativity is conducted using a device akin to the BCI. This device serves to convert brain signals into commands for an external device, enabling the execution of the given command. The present technique diverges from the traditional BCI in that it converts electrical brain signals into either a light or digital spectrum, instead of translating them directly into commands for external devices. The sensors employed in this context may resemble those utilized in generalized brain-computer interface devices or those employed in lie detectors. The BCI detects a type of emitted energy derived from brain activity measurement, in that the measured energy originates specifically from the electrical activity emitted by the region of the brain associated with creativity, rather than encompassing all brain cells.

The methodology for differentiating students into “gifted” and “non-gifted” groups begins by measuring the quantity and speed of changes of electrical energy emitted from brain activity during cognitive processes using a sensor. The sensed outputs from the BCI are converted from emitted electrical energy into light waves, which manifest as a color spectrum displayed on a monitor screen or as a digital output. The quantity and speed of changes of electrical energy emitted by a group of students (including those classified as gifted, average, and below average) are measured based on conventional intelligence tests. Subsequently, the device is then programmed to categorize the BCI outputs based on models of students' cases, such as by way of non-limiting example, the above categories of “gifted,” “average,” and “below average”. As a result, the device can make judgments regarding students based on the quantity and speed of changes of electrical energy emitted from the specific area of the brain associated with creativity while the student is engaged in problem-solving activities. Students are then classified based on the pre-programmed data within the device. To ensure adaptability to varying environments and different stages of student development, the programming of the device can be updated accordingly. This enables the device to accommodate changes in the learning environment and the evolving needs of students as they progress through different age groups.

### Closing Statement

It will be understood that many additional changes in the details, materials, steps and arrangement of parts, which have been herein described and illustrated to explain the nature of the subject matter, may be made by those skilled in the art within the principle and scope of the invention as expressed in the appended claims.

1. A method for identifying and categorizing people according to educational capability as indicated by detected brain activity, the method comprising:
providing a Brain-Computer Interface (BCI) and associated sensor electrodes;
positioning the sensor electrodes on a subject's head at positions to allow a detection of brain electrical activity at said locations;
providing an output of the BCI to a monitor;
monitoring the subject's brain activity emanating from the subject's brain with the BCI connected to the sensor electrodes; and
sensing quantity, speed of transmission, and speed of change measurements of brain electrical activity emanating from the subject's brain using the BCI, monitoring the sensed quantity and speed of change measurements and displaying the monitored sensed quantity, speed of transmission, and speed of change measurements as a color spectrum where the sensed quantity, speed of transmission, and speed of change measurements are measured based upon a subject performing conventional intelligence tests.

2.-4. (canceled)

5. Apparatus for identifying and categorizing people according to educational capability as indicated by detected brain activity, comprising:
a Brain-Computer Interface (BCI) and associated sensor electrodes, wherein placement of the sensor electrodes at locations on a subject's head suitable for detection of brain electrical activity emanating from a brain region and positioning the sensor electrodes on the subject's head allows the detection of the brain electrical activity at said locations; and
a monitor receiving an output of the BCI,
wherein monitoring the subject's brain activity with the BCI connected to the sensor electrodes and sensing quantity, speed of transmission, and speed of change measurements of brain electrical activity emanating from the brain region using the BCI allows for monitoring a sensed quantity, a sensed speed of transmission, and a sensed speed of change measurement and using the sensed quantity, sensed speed of transmission, and sensed speed of change measurements to categorize the subject according to gifted and non-gifted categories based on pre-programmed data associated with student models and the quantity of emitted energy as detected by said monitoring the sensed quantity, the sensed speed of transmission, and the sensed speed of change measurements and displaying the monitored sensed quantity, the sensed speed of transmission, and the sensed speed of change measurements as a color spectrum where the sensed quantity, the sensed speed of transmission, and the sensed speed of change measurements are measured based upon a subject performing conventional intelligence tests.

6. (canceled)
